(19) United States
(12) Patent Application Publication
(10) Pub. No.: US 2006/0093647 A1
(43) Pub. Date: May 4, 2006

(54) Multiple layer coating composition

(76) Inventors: Manuel A. Villafana, Plymouth, MN (US); John L. Babitt, Minnetonka, MN (US); Jacquie M. Campbell, Minneapolis, MN (US); Alex Alden Peterson, Maple Grove, MN (US)

Correspondence Address: Haugen Law Firm PLLP, Suite 1130, 121 South Eighth Street, Minneapolis, MN, 55402, US

(21) Appl. No.: 10/977,308

(22) Filed: Oct. 29, 2004

(51) Int. Cl. A61K 31/4745; A61K 31/337; A61F 2/00

(52) U.S. Cl. 424/426; 427/2.1; 427/487; 514/291; 514/449

(57) ABSTRACT

A multiple layer coating for application upon surfaces of an implantable medical device, a first layer of such coating includes an anti-thrombogenic factor and a second layer of such coating includes a hyperplasia inhibiting factor that is releasably contained therein. The hyperplasia inhibiting factor is controllably released from the multiple layer coating over a predetermined period of time upon implantation of the medical device in vivo.

### FIELD OF THE INVENTION

The present invention relates to medical device coatings generally, and more particularly to a multi-functional coating defined in a plurality of distinct layers. The combination coating of the present invention includes a biocompatible material and a bioactive material.

### BACKGROUND OF THE INVENTION

An increasing variety of surgical procedures now involve the implantation of medical devices into the human body, whether such implantation is temporary or permanent. A particularly burgeoning medical field in which the implantation of artificial devices has proven to be invaluable is in coronary surgical procedures, though a variety of other medical fields also utilize implantable medical devices.

The implantation of such medical devices, however, introduces risks of complications related to having a foreign body surgically affixed within the patient. Such complications include, for example thrombogenic action caused by the foreign body response of the patient which can lead to fibrous deposition on device surfaces, and the wound healing response of the body acting at the surgical implantation site, which response can result in hyperplasia.

In an effort to minimize the likelihood of such complications, coatings have been developed for application to surfaces of the respective medical devices exposed to the body of the human patient when positioned in vivo. Typically, such coatings have focused on masking the device to render it more biocompatible and/or providing biochemical activity to the device surfaces to resist or suppress the thrombogenetic response of the patient. Such biochemical materials include, for example, heparin, albumin, and streptokinase.

Another approach to minimizing the adverse affects of implantation of medical devices is in delivering bioactive components to the surgical implant location, primarily to inhibit hyperplasia at the surgical site. Recently, incorporation of anti-proliferative drugs as hyperplasia inhibiting agents have been incorporated into coatings placed upon implantable medical devices, such as coronary stents. Such coatings act to release the anti-proliferative drugs over time from a platform immediately adjacent to the surgical site, such that the pharmaceutical compounds have the most beneficial anti-hyperplasia effect possible. Typically, the release of such anti-proliferative drugs is enabled through the utilization of biodegradable or bioreusable polymers forming the matrix of a drug-containing coating that is applied to surfaces of the implanted device ultimately positioned adjacent to internal trauma caused by the surgical implantation procedure, and in the local area where the body tries to isolate foreign bodies that may not have caused trauma during implantation.

Though the coating variants described above have been individually implemented with some success, certain device applications find the individual use of such coatings to be ineffective, expensive, or both.

It is therefore a principal object of the present invention to provide a coating combination of a plurality of distinct coating layers incorporating both biocompatibility and bioactivity characteristics.

It is a further object of the present invention to provide a multiple layer coating combination for use in connection with implantable medical devices, which multiple layer coating combination incorporates both an anti-thrombogenetic factor and a hyperplasia inhibiting factor.

It is another object of the present invention to provide a multiple layer medical device coating having an anti-thrombogenic factor and a hyperplasia inhibiting factor that is released adjacent to the surgical site over a pre-determined period of time.

It is a still further object of the present invention to provide a method for curing an ultraviolet radiation-curable coating through the use of an ultraviolet radiation transmitting fiber-optic device operably placed in proximity to the ultraviolet radiation-curable coating for a period of time sufficient to cure the coating.

### SUMMARY OF THE INVENTION

By means of the present invention, a multiple factor coating combination is provided for application upon implantable medical devices. The coating combination of the present invention provides an efficient and reliable vehicle for releasing a bioactive drug over a predetermined period of time from a biocompatible platform adjacently positioned with respect to the surgical implantation site. Such proximity to the surgical implantation site reduces the total dosage of the bioactive drug needed to be released into the patient's body. As such, the coating combination of the invention is more efficient, less expensive, and potentially less harmful than general delivery of the bioactive drug.

In a particular embodiment, the coating combination of the invention includes a first layer including an anti-thrombogenic factor, and a second layer having a hyperplasia inhibiting factor that is controllably released therefrom over a predetermined period of time. Preferably, at least about 80% of a predefined dosage of the hyperplasia inhibiting factor contained in the second layer is controllably released within 90 days of implantation.

In embodiments where hyperplasia inhibiting factor is critical only at designated portions of the associated medical device, the second layer is preferably overlaid upon a portion of the first layer.

### DETAILED DESCRIPTION OF THE PREFERRED EMBODIMENTS

The objects and advantages enumerated above together with other objects, features, and advances represented by the present invention will now be presented in terms of detailed embodiments described with reference to specific examples which are intended to be representative of various possible embodiments of the invention. Other aspects and embodiments of the invention are recognized as being within the grasp of those having ordinary skill in the art.

The coating combination of the present invention is preferably applied to surfaces of medical devices that, when implanted, are exposed to the patient's body. Such devices include, for example, coronary stents, mechanical heart valves, vascular connector members such as those described in U.S. Pat. Nos. 6,241,761 and 6,241,764, and the like.

The coating combination of the present invention is preferably made up of at least first and second layers each incorporating into the coating combination biocompatible and/or bioactive materials. For example, a first layer of the coating combination includes an anti-thrombogenic factor such as heparin, albumin, streptokinase, urokinase, or tissue plasminogen activator (TPA). Such an anti-thrombogenetic factor may also be formed of combinations of different materials, such as those described above.

The first layer of the coating combination of the present invention may be formulated in accordance with that described in, for example, U.S. Pat. Nos. 4,973,493 and 4,979,959, the contents of which are herein incorporated by reference. The disclosures of the patents incorporated by reference above describe example formulations and methodologies for covalently bonding an anti-thrombogenic factor to the surface of, for example, an implantable medical device. In some embodiments, the first layer may be made up of a base coat and a top coat, which together are covalently bonded to a Parylene tie-layer coating disposed upon respective surfaces of the medical device. Moreover, the first layer preferably includes at least one photo-activatable cross-linking agent for assisting in covalently bonding the anti-thrombogenic factor to the Parylene tie-layer coating and/or directly upon the surfaces of the medical device. Examples of cross-linking agents and tie-layer coatings useful in the coating combination of the present invention are described in U.S. Pat. Nos. 5,002,582; 5,512,329; 6,077,698; 6,278,018; 6,603,040; and 6,706,408, the contents of which are herein incorporated by reference.

The second layer of the coating combination of the present invention preferably includes a hyperplasia inhibiting factor that is controllably released therefrom over a pre-determined period of time. Such a hyperplasia inhibiting factor may be selected from a variety of materials or combinations thereof, with specific examples of such a factor including paclitaxel, everolimus and sirolimus. Paclitaxel, for example, is available from several FDA approved vendors such as Mayne Pharma, Inc. of Denver, Colo. while everolimus is available from Novartis of Basel, Switzerland, and sirolimus is available Bristol-Myers Squibb, Inc. of New York, N.Y.

The second layer incorporating a hyperplasia inhibiting factor that is controllably released over time is described in, for example, U.S. Pat. Nos. 6,214,901 and 6,344,035, the contents of which are herein incorporated by reference. Most preferably, the second layer coating is formulated from a solution having polybutylmethacrylate, polyethelenevinyl acetate, and the hyperplasia inhibiting factor dissolved in chloroform, with such solution being coated and cured on the implantable medical device.

The second layer is preferably formulated so as to controllably release about 80% of the designated hyperplasia inhibiting factor dosage therefrom within 90 days of implantation of the coated medical device in vivo. For hyperplasia inhibiting factor release characteristics, it has been found that the second layer is preferably overlaid upon a portion of the first layer, such that the covalent bonding described with reference to the first layer does not interfere with the controlled time release of the hyperplasia inhibiting factor from the second layer.

### EXAMPLE

A titanium vessel connector tube segment having an outside diameter of 2 mm was treated with a Parylene tie-layer coating composition (Parylene is a trademark of the Union Carbide Corporation), which includes silane in a 50% by weight isopropyl alcohol/deoinized water solution. The tubing segment was soaked in the silane solution in a clean room having a relative humidity held below 60%. The Parylene coating was gas deposited in a thin, conformal, polymer coating of about 1-2 μm thickness on all surfaces of the tubing segment.

A basecoat solution of polyvinylpyrrolidone (PVP) polymer available from SurModics, Inc. of Eden Prairie, Minn. under the chemical identifier PV05 in isopropyl alcohol at a concentration of 5 mg/ml was prepared. The Parylene-treated tubing segment was dipped into the basecoat solution for 5 seconds and then withdrawn from the basecoat solution at a rate of 1 cm/sec to ensure an even coat, after which the coated tubing segment was air-dried for 30 minutes at room temperature. Once dried, the tubing segment was continuously rotated within an ultraviolet radiation chamber for 3 minutes. The basecoat was further cured by exposure to a spotlight ultraviolet radiation source for 60 seconds.

A topcoat solution was prepared by mixing a first PVP polymer available from Surmodics under the chemical identifier PV01, a second PVP polymer available from Surmodics under the chemical identifier PR05, a photo-activatable cross-linking agent available from Surmodics under the chemical identifier PR04, and modified heparin having photo-activatable groups molecularly bound thereto and available from Surmodics under the chemical identifier HP01, with the mixture being solvated in a 40% isopropyl alcohol/deionized water solution. The PV01 polymer was present at a concentration of 7 mg/ml, the PV05 polymer was present at a concentration of 3 mg/ml, the PR04 photo-activatable linking agent was present at a concentration of 0.4 mg/ml, and the modified heparin was added at a concentration of 4 mg/ml of total solution.

The tubing segment was then dipped into the topcoat solution using the same procedure as that performed with the basecoat solution. The coated tubing segment is then air-dried for 1 hour at room temperature. The topcoat is then cured through exposure to ultraviolet radiation in the same procedure as that performed to cure the basecoat on the tubing segment. The combined basecoat and topcoat thickness was measured at about 3 μm, and had an active heparin concentration of 14 mU/cm².

A further coating solution was prepared by mixing 65% by weight polybutylmethacrylate available from Aldrich Chemical Company, Inc.; 25% by weight polyethelenevinylacetate available from Aldrich Chemical Company, Inc.; and 10% by weight paclitaxel available from Mayne Pharma, Inc., together in a 100% chloroform solvent. The resultant solution was spray-coated onto the tubing segment at a relative humidity of 30%, and subsequently air-dried for 30 minutes. The thickness of the spray coat was about 4 μm, with a target final paclitaxel concentration of 1.6 μg/mm².

The coated tube segment was implanted in 6 pigs as a vessel connecting (vc) device for a 30-60-90 day evaluation trial period. Upon completion of the trial, the pigs were inspected for evidence of thrombogenetic invasion and hyperplasia. No signs of either condition were reported.

Though the tube segment was coated through the specifically-identified methodologies as described above, it is to be understood that other coating techniques such as brush, dauber, ultrasound, microfine spraying, vacuum deposition, and the like may be utilized instead of those described with reference to the example above. Such coating techniques are known in the art, and one of ordinary skill would be expected to recognize the various coating techniques available to apply the coatings of the present invention given the disclosure hereof.

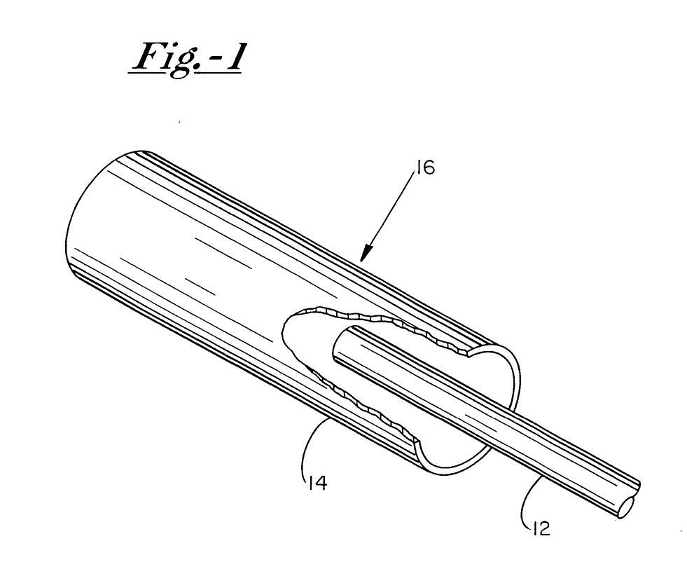

An ultraviolet radiation curing technique utilizing a fiber optic lead is illustrated in FIG. 1, with fiber optic lead 12 being operably coupled to an ultraviolet radiation source (not shown), and arranged such that ultraviolet radiation is emitted out through at least a portion of lead 12. To cure the respective coating disposed on inner surface 14 of tube segment 16, fiber optic lead element 12 is inserted into tube segment 16 to thereby be adjacently positioned with respect to coated surface 14 of tube segment 16. In preferred embodiments, fiber optic lead 12 is drawn through tube segment 16 or otherwise placed in adjacent relationship with the surface to be cured such that ultraviolet radiation emitted from fiber optic lead element 12 impinges upon every portion of the coated surface for at least about 3 minutes. Such a time period has been found by the applicant to sufficiently cure the coatings of the present invention.

The invention has been described herein in considerable detail in order to comply with the patent statutes, and to provide those skilled in the art with the information needed to apply the novel principles and to formulate and use embodiments of the invention as required. However, is to be understood that various modifications of the invention can be accomplished without departing from the scope of the invention itself.

What is claimed is:

1. A multiple layer coating for application upon surfaces of an implantable medical device, a first layer of said coating including an anti-thrombogenic factor, and a second layer of said coating including a hyperplasia inhibiting factor that is releasably contained therein, said hyperplasia inhibiting factor being controllably released over a predetermined period of time upon implantation of said medical device in vivo.

2. A multiple layer coating as in claim 1 wherein said anti-thrombogenic factor is applied to respective surfaces of said medical device at a concentration of between about 5 and 25 mU/cm².

3. A multiple layer coating as in claim 1 wherein said hyperplasia inhibiting factor is contained in said second layer at a concentration of between about 2 and 30% by weight of said second coating layer.

4. A multiple layer coating as in claim 1 wherein a pre-designated dosage of said hyperplasia inhibiting factor is applied to respective surfaces of said medical device at a concentration of between about 0.5 and 10 μg/mm².

5. A multiple layer coating as in claim 1 wherein said hyperplasia inhibiting factor is selected from the group consisting of paclitaxel, everolimus, and sirolimus.

6. A multiple layer coating as in claim 1 wherein at least about 80% of the designated hyperplasia inhibiting factor dosage is operably released from said second layer of said coating within 90 days of implantation of said medical device in vivo.

7. A multiple layer coating as in claim 1 wherein said second layer is overlaid upon a portion of said first layer.

8. A method of curing and ultraviolet radiation curable coating disposed on respective inner surfaces of a substantially hollow medical device, said method comprising:
(a) providing a fiber optic device that is operably coupled to an ultraviolet radiation source, and operably arranged such that ultraviolet radiation is axially directed through said fiber optic device; and
(b) positioning said fiber optic device adjacent to respective said coated surfaces of said medical device in such a manner so that ultraviolet radiation emitted from said fiber optic device impinges upon said coated surfaces for a period of time sufficient to cure said coating.

9. A method as in claim 8 wherein said period of time is at least about 3 minutes.
